(19) United States
(12) Patent Application Publication
(10) Pub. No.: US 2017/0049987 A1
(43) Pub. Date: Feb. 23, 2017

(54) MOISTURE TRAP ASSEMBLY FOR RESPIRATORY CIRCUI

(71) Applicant: Brooke Higginbotham, Las Cruces, NM (US)

(72) Inventor: Brooke Higginbotham, Las Cruces, NM (US)

(21) Appl. No.: 15/243,575

(22) Filed: Aug. 22, 2016

Related U.S. Application Data
(60) Provisional application No. 62/208,718, filed on Aug. 23, 2015.

(51) Int. Cl. A61M 16/08; A61M 16/16

(52) U.S. Cl. CPC A61M 16/0808; A61M 16/0875; A61M 16/0816; A61M 16/16; A61M 2202/0208

(57) ABSTRACT

A moisture trap assembly for a respiratory circuit comprises a flow connector unit comprising an inlet tube in fluid connection with an outlet tube and an annular lid comprising an air tight seal. The inlet and outlet tubes are adapted to fit the respiratory circuit. A receptacle is removably attached to the annular lid for collecting condensed moisture, wherein the receptacle comprise an actuator member configured to actuate the air tight seal of the annular lid upon attachment.

### CROSS REFERENCE TO RELATED PATENT APPLICATIONS

This patent application claims the priority and benefit, under 35 U.S.C. §119(e), of U.S. Provisional Patent Application Ser. No. 62/208,718 filed Aug. 23, 2015, entitled “Moisture Trap Assembly for Respiratory Circuit.” U.S. Provisional Patent Application Ser. No. 62/208,718 is herein incorporated by reference in its entirety.

### TECHNICAL FIELD

Embodiments are generally related to respiratory devices. Embodiments are further related to methods and systems associated with respiratory circuits. More particularly, embodiments are related to a moisture trap assembly for a respiratory circuit. Certain embodiments relate to a medical utility device for use with respiratory airline apparatus comprising a moisture or water vapor trap device for capturing moisture from the respiratory air or breathing gas passing through a respiratory circuit.

### BACKGROUND

People with respiratory ailments, including, but not limited to, general respiratory distress, respiratory syncytial virus (RSV), congestive heart failure (CHF), asthma, pneumonia, chronic obstructive pulmonary disorder (COPD), patients in neonate care, adult intensive care, sub-acute care, and palliative care, who are in need of high pressure flow medical apparatuses to prevent intubation or to help aid with the normal breathing process, may utilize a respiratory circuit or respiratory airline system such as a high flow oxygen (HFO) apparatus.

A respiratory airline apparatus is a widely used apparatus in the medical field for facilitating patients' breathing by providing a continuous supply of clean breathing gas, such as oxygen. In addition, the breathing system is usually used in combination with a humidifier in order to adjust the humidity of the breathing gas according the patient's body temperature, thereby raising the comfort level during breathing. The breathing system is also capable of dosing respiratory medication to further augment the patient's breathing circulation.

In a low temperature environment, the breathing gas and/or patient's exhaled breath in the respiratory circuit tends to condense inside the circuit of the breathing system. Thus, several liquid trap assemblies are normally provided to collect the waste liquid accumulated in the circuit. For example, liquid trap assemblies can be provided at each of the following outlets: the humidifier, the patient's oral/nasal area, and the gas cylinder. The waste liquid in the liquid trap assemblies must be drained regularly in order to properly maintain the function of the breathing system, and to prevent microbial contamination.

The condensation and accumulation of water, caused by the respiratory airline apparatus, has been noted by healthcare professionals nationwide, and by the United States Food and Drug Administration (FDA). According to the United States Food and Drug Administration (FDA), the airline circuit system, known as a Vapotherm, can accumulate water at an average rate of 3 milliliters/hour. This rate is comparable to the accumulation of water observed in healthcare facilities during use of the Vapotherm.

Accumulation of moisture or water vapor is a serious concern in the respiratory airline delivery systems. Such condensation can result in water accumulation or “raining out.” Condensation can also cause water to flow into the patient's nostrils. Without a moisture trap apparatus, accumulation of water in the aerosol adapter can cause significant rain out which can lead to further complications.

Prior art solutions are fraught with drawbacks. Limitations of such prior art include an inability to connect accurately with the input and output lines of the aerosol adapter and/or respiratory circuit such as Vapotherm apparatus and obstruction to the flow of heliox or precision air due to frequent disconnection of the respiratory circuit system from the aerosol adapter, which results in both water leakage and air flow obstruction. During any time of disconnect, the patient is not receiving high flow therapy. Therefore, prior art approaches do not effectively solve the problems associated with water accumulation and obstructive air flow in high flow apparatuses.

Accordingly, there is a need in the art for methods, systems, and devices that provide a moisture trap for a respiratory circuit that adequately addresses the problems associated with water accumulation and obstructive airflow as detailed herein.

### BRIEF SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is therefore one aspect of the disclosed embodiments to provide a moisture trap assembly for a respiratory circuit.

In another aspect of the disclosed embodiments the moisture trap can comprise a flow connector unit comprising an inlet tube in fluid connection with an outlet tube and an annular lid comprising an air tight seal.

In another aspect of the disclosed embodiments the inlet and outlet tubes are adapted to fit to the respiratory circuit of a high flow oxygen apparatus.

In another aspect of the disclosed embodiments a receptacle is removably attached to the annular lid for collecting condensed moisture. The receptacle can comprise an actuator configured to actuate the air tight seal of the annular lid upon attachment.

The aforementioned aspects and other objectives and advantages can now be achieved as described herein. In one example embodiment, a moisture trap system comprises an inlet tube in fluid connection with an outlet tube, an annular lid comprising an airtight seal, and a receptacle removably attached the annular lid. The system can further comprise a patient circuit of a respiratory airline system wherein the moisture trap assembly is inline in the patient circuit

### BRIEF DESCRIPTION OF THE FIGURES

The accompanying figures, in which like reference numerals refer to identical or functionally-similar elements throughout the separate views and which are incorporated in and form a part of the specification, further illustrate the embodiments and, together with the detailed description, serve to explain the embodiments disclosed herein.

FIG. 1A depicts a flow connector unit associated with a moisture trap, in accordance with an example embodiment;

FIG. 1B depicts flow connector unit associated with a moisture trap, in accordance with an example embodiment;

FIG. 2A depicts a moisture trap, in accordance with an example embodiment;

FIG. 2B depicts a section view of a moisture trap in line with a respiratory airline apparatus, in accordance with an example embodiment;

FIG. 3A depicts a perspective view of flow connector unit showing exemplary dimensions, in accordance with an example embodiment;

FIG. 3B depicts a side view of flow connector unit showing exemplary dimensions, in accordance with an example embodiment;

FIG. 3C depicts a bottom view of flow connector unit showing exemplary dimensions, in accordance with an example embodiment;

FIG. 3D depicts is a sectional view of flow connector unit showing exemplary dimensions, in accordance with an example embodiment; and

FIG. 4 depicts a flow chart illustrating steps in a method for collecting condensation from a respiratory circuit, in accordance with an example embodiment.

### DETAILED DESCRIPTION

Subject matter will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of systems, apparatuses, methods, or any combination thereof (other than software per se). The following detailed description is therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment, and the phrase “in another embodiment” as used herein does not necessarily refer to a different embodiment. It is intended, for example, that claimed subject matter include combinations of example embodiments in whole or in part.

In general, terminology may be understood, at least in part, from usage in context. For example, terms, such as “and,” “or,” or “and/or” as used herein may include a variety of meanings that may depend, at least in part, upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be utilized to describe any feature, structure, or characteristic in a singular sense or may be utilized to describe combinations of features, structures or characteristics in a plural sense. Similarly, terms such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The embodiments disclosed herein provide moisture trap systems and methods configured to capture water at equal to or greater than 70 cc. This allows one moisture trap collection cup to accumulate water for approximately 23 hours prior to changing the removable and disposable cup for a replacement cup.

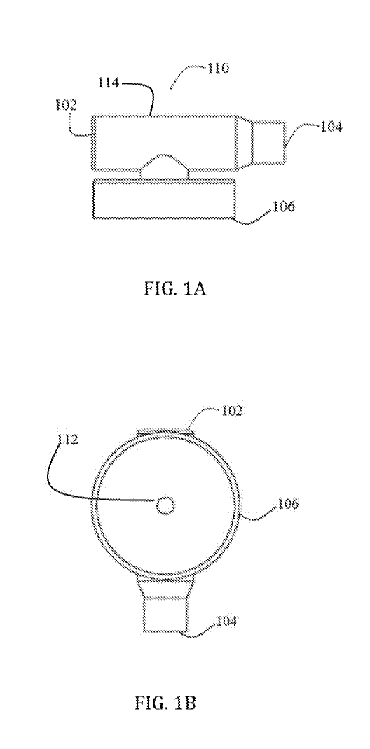

Referring to FIG. 1A, a flow connector unit 110 associated with a moisture trap assembly 100 is illustrated in accordance with an embodiment. The flow connector 110 comprises an inlet tube 102 in fluid connection with the outlet tube 104. The inlet tube 102 and outlet tube 104 preferably comprise a hollow conduit, with their ends adapted to fit, in line, to a respiratory circuit.

For example, in an embodiment, the dimensions of inlet tube 102 and outlet tube 104 are designed to fit accurately with the patient delivery tube of a respiratory circuit associated with an HFO apparatus and an aerosol adapter respectively. The flow connector 110 further comprises an annular lid 106 extending generally downwards from the conduit connecting the input tube 102 and output tube 104. The annular lid 106 provides an airtight seal in the middle of the lid 106.

FIG. 1B, shows a bottom view of the annular lid 106 of the flow connector unit 110. The annular lid 106 is connected to the inlet 102 and outlet tube 104 via a narrow neck.

The annular lid also provides an airtight seal 108. In an embodiment, the airtight seal 108 can comprise a rubber stopper configured to plug an opening or drain 112 in the annular lid 106. The rubber stopper forms an airtight seal so that liquid and gas cannot escape through the drain 112 unless the rubber stopper is moved. In an embodiment, the airtight seal 108 may further comprise a tensioning device to securely hold the rubber stopper in the drain 112 in annular lid 106.

The middle conduit section 114 between the inlet tube 102 and outlet tube 104 can further comprise a channel or opening allowing fluid communication between the inlet tube 102 and outlet tube 104. The middle conduit section 114 is configured with a slight downward slanting angle on either side of the narrow neck, in order to encourage the accumulated water to flow towards the opening of the airtight seal 108.

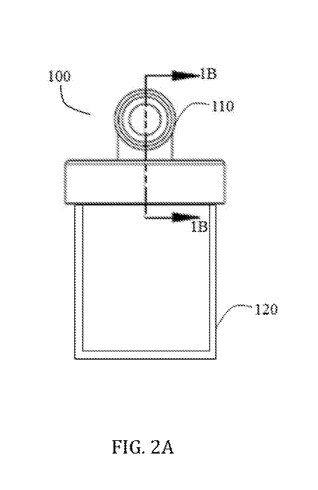

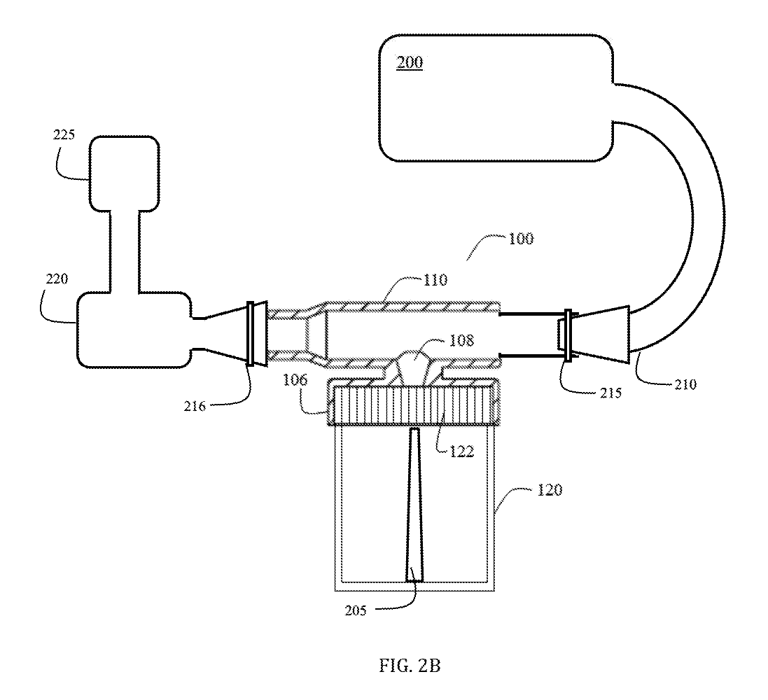

FIG. 2A shows a perspective view of the moisture trap assembly 100 according to an embodiment of the present invention. The moisture trap assembly 100 comprises a flow connector unit 110 attached to a receptacle 120, for collecting the water accumulated from trapped moisture or water vapor inside the flow connector 110. The receptacle 120 can be removed after water has collected inside. The water can be drained, the receptacle cleaned and then the receptacle 120 can be reattached to the annular lid.

FIG. 2B shows a sectional view of the moisture trap assembly 100 in line in a patient circuit associated with a respiratory airline apparatus 200 according to an embodiment. The moisture trap assembly 100 can comprise a flow connector unit 110 attached to the receptacle 120.

The receptacle 120 can be removably attached to the annular lid 106, with use of threads on the outer surface of receptacle mouth 122, which are received by the threads on inner circumference of the annular lid 106. An airtight seal 108 is positioned in the middle of the annular lid 106. The air tight seal 108 can comprise a rubber stopper that seals the drain 112 generally formed in the annular lid 106 between the inlet tube 102 and the outlet tube 104. The middle conduit section 114 comprises downward slanting inner walls in order to guide water into the attached receptacle 120 through drain 112.

Without receptacle 120 attached to the flow connector unit 110, the rubber stopper fits in the drain 112 creating an airtight seal 108. This represents a closed position of airtight seal 108. When airtight seal 108 is respiratory air and/or breathing gas passes smoothly between the inlet tube 102 and outlet tube 104, thereby preventing any loss of respiratory air or gas.

The airtight seal 108 is actuated to an open position when the receptacle 120 is attached to the flow connector unit 110. In an embodiment, the receptacle 120 may include an actuating member 205 configured to actuate the seal 108 from a closed to open position upon attachment of the receptacle 120 to the annular lid 106, thus allowing the water accumulated from trapped moisture in flow connector 110 to trickle down through the narrow neck and drain 112 and flow into the receptacle 120.

In an embodiment, the actuating member 205 comprises a shaft affixed to the bottom of the receptacle 120 that is slightly longer than the receptacle 120. When the receptacle 120 is threaded into full engagement with the flow connector unit 110, the shaft of actuating member 205 pushes the rubber stopper up leaving the drain 112 open. In the open position, water can drip though drain 112 into receptacle 120.

When the receptacle 120 is unthreaded from the flow connector 110, the actuating member 205 is disengaged from the rubber stopper. The robber stopper resumes its natural position plugging drain 112 and the airtight seal 108 is closed to prevent air flow or gas flow loss through the airtight seal 108.

The moisture trap assembly 100 is designed to fit in line with the patient circuit of a respiratory airline apparatus 200. The respiratory airline apparatus may comprise any apparatus that provides a continuous supply of clean breathing gas, such as oxygen. The respiratory airline apparatus 200 may include a humidifier in order to adjust the humidity of the breathing gas. The breathing may also be capable of providing respiratory medication to further augment the patient's breathing circulation. In an embodiment, the respiratory airline apparatus may comprise a Vapotherm.

In an embodiment, the supply of clean breathing gas is supplied via a patient delivery tube 210. The patient delivery tube 210 can be connected to the input tube 102 of the flow connector 110. External rubber banding 215 can be used to provide a snug fit between the patient delivery tube 210 and input tube 102. The output tube 104 of the flow connector 110 can attach to aerosol adaptor 220. External rubber banding 216 can be used to provide a snug fit between the aerosol adaptor 220 and output tube 104. Cannula 225 is connected to the aerosol adaptor. Cannula 225 can comprise the tubing and ports that ultimately supply the air, gas, and/or respiratory medication to the patient.

It should be understood that the inline arrangement illustrated in FIG. 2B is exemplary. In other embodiments, the moisture trap assembly may be inserted in the patient circuit of a respiratory airline apparatus at other points. In such cases, the sizes and shapes of the input port 102 and output port 104 may be modified in order to properly fit.

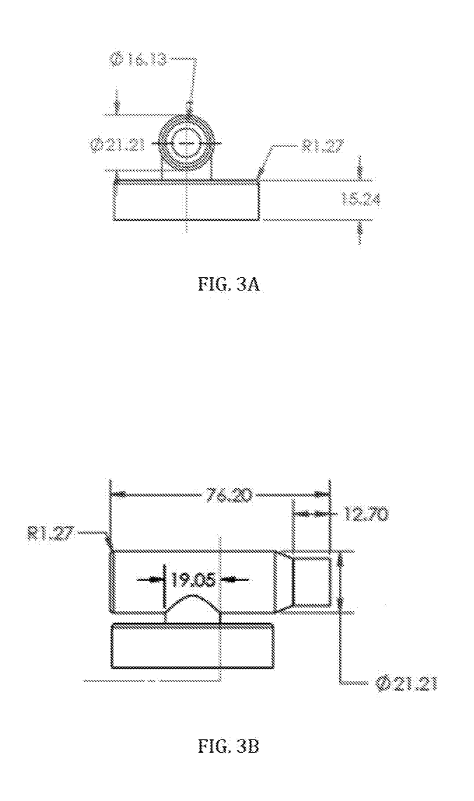

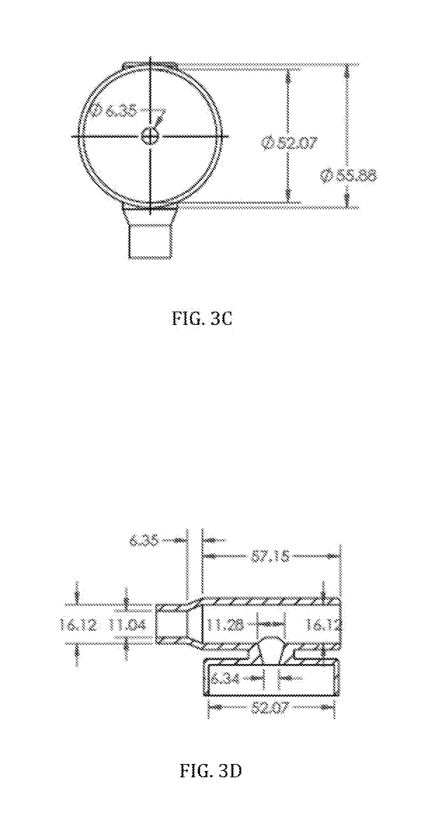

FIG. 3A-3D illustrates different views of the flow connector unit 110 showing exemplary dimensions according to an embodiment of the invention. It should be understood that the various dimensions provided in FIGS. 3A-3D are only exemplary and that other dimensions may alternatively be necessary. FIGS. 3A-3D are not meant to limit the scope of the embodiments solely to such dimensions.

In an embodiment, the inlet and outlet tubes of the flow connector unit are of a sized to engage with Vapotherm input and output dimensions. For example inlet tube 102 can have a diameter of 16.002 mm, congruent with the input tube of the respiratory circuit and outlet tube 104 can have a diameter of 10.16 mm which is 0.762 mm less than the output tube of the aerosol adapter, providing an accurate and secure fit. The outlet tube 104 can further comprise rubber banding 216, which can be, for example, approximately 0.762 mm rubber banding around the external edging of outlet tube 104 in order to provide a snug fit with the aerosol adapter 220. The rubber banding 216 prevents disconnection and creates a total output tubing of approximately 10.922 mm, which is congruent to the input tubing of the aerosol adapter.

In addition, the inlet and outlet pipes may be slightly tapered along their respective lengths to ensure a snug fit with the respiratory circuit tubing and/or aerosol adapter. The inlet and outlet tubes are linearly shaped with no slanting from the top outer ends toward the center of the lid location. The annular lid comprises a downwardly extending annular skirt consisting threads on the inner circumference for engaging with the threads of the receptacle, which can be unscrewed and separated during draining and cleaning.

In an embodiment, the receptacle 120 comprises a moisture collection cup with a capacity to contain approximately 70 cc of water. However, the cup volume may vary depending on design considerations. The receptacle 120 can be secured to the flow connector 110 unit via annular lid 106. The receptacle 120, when fully engaged, is configured to prevent the leakage of gas, water, or medication to leak from within.

In an embodiment, the collection cup may also comprise a downwardly extended funnel and a port at the bottom of the cup allowing the release of accumulated water from the flow connector unit 110. When water has accumulated in the collection cup, the cup can be unscrewed, emptied, dried and fitted again to the flow connector. During detachment of the cup, the airtight seal comes to a closed position in order to secure the air or gas flowing through the flow connector thus preventing loss of air or respiratory gas. Once the collection cup is securely attached again, the airtight seal will be actuated to an open position, thus allowing flow of accumulated water into the collection cup.

In an embodiment, the moisture trap assembly used with respiratory apparatuses is specifically designed to connect to the input tube of the Vapotherm respiratory circuit and the output tube of the Vapotherm aerosol adapter by providing congruent dimensions and external rubber banding in the outlet tube of moisture trap assembly. The rubber banding at the inlet and outlet tubes provide a snug fit, thus preventing disconnection and leakage of water into the patient respiratory line or other equipment.

The moisture trap assembly 100 can be configured to attach accurately and easily to the Vapotherm respiratory circuit, or to any other respiratory apparatuses. The moisture trap assembly 100 reduces the accumulation of water in the respiratory apparatus, thereby improving patient comfort. The moisture trap provides a less compromised flow of heliox, precision flow, or other gas by providing an avenue for water accumulation while also allowing the uninterrupted passage of high pressure heliox and oxygen flow to the patient.

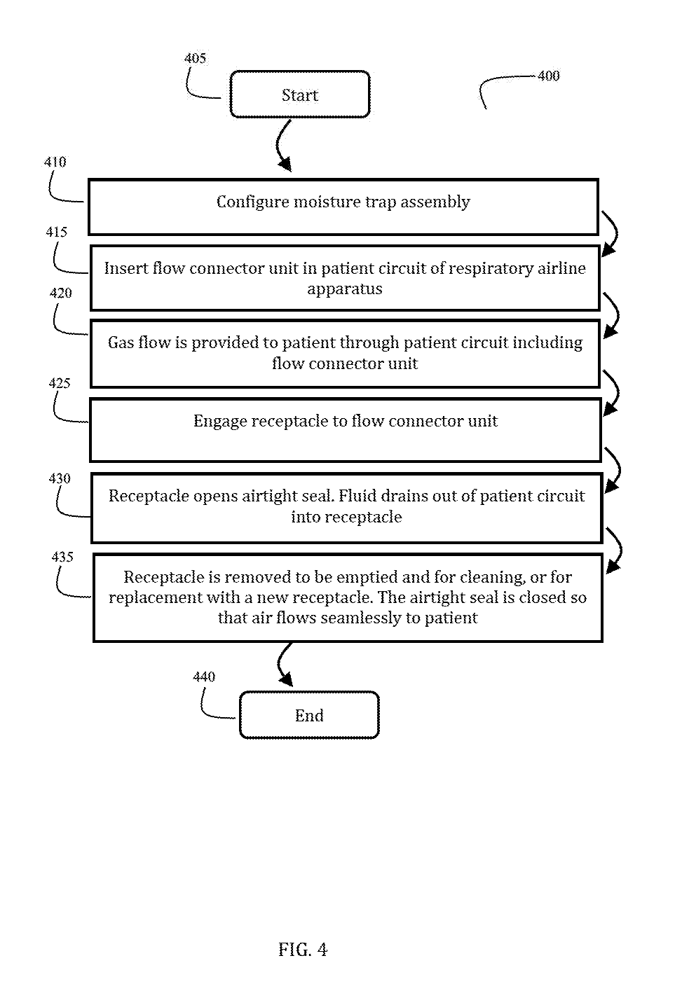

FIG. 4 illustrates a flow chart illustrating steps in a method 400 for collecting condensation from a respiratory circuit. The method begins at 405. At block 410 a moisture trap assembly can be configured to include an input and output tube and an annular lid with an air tight seal. A receptacle for collecting liquid can be included. At block 415, the moisture trap assembly can be inserted into the flow path of a patient circuit associated with a respiratory airline apparatus. In an embodiment, the respiratory airline apparatus can be a Vapotherm but other respiratory airline apparatuses may alternatively be used. The connections between the patient circuit and the flow connector can be secured with rubber banding.

Gas flow can now be provided through the patient circuit as shown at block 420. Gas can flow smoothly through the flow connector unit because the airtight seal prevents leakage. At block 425 a collection receptacle can be engaged with the flow connector unit. The collection receptacle engagement is also airtight and does not alter gas flow to the patient. At block 430, an actuating member in the receptacle opens the airtight seal. In an embodiment, the actuating member comprises a shaft that removes a rubber stop from a drain in the annular lid of the flow connector. Fluid that has accumulated, or is accumulating in the patient circuit flows naturally through the drain and into the collection receptacle. The flow is facilitated by inner walls that slope towards the drain in the flow connector unit.

When the receptacle is filled to capacity or requires cleaning or replacement, it is removed from the flow connector unit as shown at block 435. Removal of the receptacle automatically doses the airtight seal so that again, air flow to the patient is not interrupted. It should be appreciated that once the moisture assembly apparatus is installed in the gas flow path of the patient circuit, steps 425, 430, and 435 may be repeated as many times as necessary. The method end at block 440.

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above and/or in the attachments, and of the corresponding application(s), are hereby incorporated by reference.

Based on the foregoing, it can be appreciated that a number of embodiments, preferred and alternative, are disclosed herein. For example, in one embodiment, a moisture trap system comprises an inlet tube in fluid connection with an outlet tube, an annular lid comprising an airtight seal, and a receptacle removably attached the annular lid. The system can further comprise a patient circuit of a respiratory airline system wherein the moisture trap assembly is inline in the patient circuit.

In an embodiment, an inlet tube is adapted to fit the patient circuit and the outlet tube is adapted to fit the patient circuit. In another embodiment, the airtight seal further comprises a drain and a stop configured to form an airtight seal in the drain. In another embodiment, the system comprises an actuating member configured to operate the airtight seal.

In an embodiment, the receptacle is configured to collect liquid in the respiratory airline system resulting from trapped moisture. In another embodiment, a banding assembly is configured to secure the inlet tube and the outlet tube to the patient circuit.

In another embodiment, an apparatus for collecting moisture comprises a patient circuit of a respiratory airline apparatus, an inlet tube in fluid connection with the patient circuit, an outlet tube in fluid connection with the patient circuit, an annular lid configured on the inlet tube and the outlet tube comprising an airtight seal, and a receptacle removably attached the annular lid.

In an embodiment, the inlet tube is adapted to fit the patient circuit and the outlet tube is adapted to fit the patient circuit. In another embodiment, the airtight seal further comprises a drain and a stop configured to form an airtight seal in the drain. In another embodiment, an actuating member is configured to operate the airtight seal. In another embodiment, the receptacle is configured to collect liquid in the respiratory circuit resulting from trapped moisture.

In yet another embodiment, a method for collecting moisture in a respiratory airline system comprises configuring a moisture trap system inline in a patient circuit of the respiratory airline system, connecting a receptacle to the moisture trap system, and collecting liquid in the respiratory circuit resulting from trapped moisture.

In an embodiment of the method, the moisture trap system comprises an inlet tube in fluid connection with an outlet tube, an annular lid comprising an airtight seal, and a receptacle removably attached the annular lid. The method may further comprise providing at least one of breathing gas and respiratory medication via the patient circuit.

In another embodiment, connecting a receptacle to the moisture trap system further comprises opening the airtight seal of the annular lid with an actuating member. In an embodiment, the method comprises removing the receptacle, emptying the receptacle, and cleaning the receptacle. In an embodiment, removing the receptacle further comprises automatically closing the airtight seal of the annular lid.

In another embodiment, the method comprises adapting the inlet tube to fit the patient circuit and adapting the outlet tube to fit the patient circuit. The method can further comprise securing the inlet tube and the outlet tube to the patient circuit with a banding assembly.

It will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following claims.

What is claimed is:

1. A moisture trap system comprising:
an inlet tube in fluid connection with an outlet tube;
an annular lid comprising an airtight seal; and
a receptacle removably attached said annular lid.

2. The system of claim 1 further comprising:
patient circuit of a respiratory airline system wherein said moisture trap assembly is inline in said patient circuit.

3. The system of claim 2, wherein said inlet tube is adapted to fit said patient circuit.

4. The system of claim 2, wherein said outlet tube is adapted to fit said patient circuit.

5. The system of claim 1 wherein said airtight seal further comprises:
a drain; and
a stop configured to form an airtight seal in said drain.

6. The system of claim 5 further comprising:
an actuating member configured to operate said airtight seal.

7. The system of claim 2 wherein said receptacle is configured to collect liquid in said respiratory airline system resulting from trapped moisture.

8. The system of claim 2 further comprising a banding assembly configured to secure said inlet tube and said outlet tube to said patient circuit.

9. An apparatus for collecting moisture comprising:
a patient circuit of a respiratory airline apparatus;
an inlet tube in fluid connection with said patient circuit;
an outlet tube in fluid connection with said patient circuit;
an annular lid configured on said inlet tube and said outlet tube comprising an airtight seal; and
a receptacle removably attached said annular lid.

10. The apparatus of claim 9, wherein said inlet tube is adapted to fit said patient circuit and said outlet tube is adapted to fit said patient circuit.

11. The apparatus of claim 9 wherein said airtight seal further comprises:
a drain; and
a stop configured to form an airtight seal in said drain.

12. The apparatus of claim 11 further comprising:
an actuating member configured to operate said airtight seal.

13. The apparatus of claim 9 wherein said receptacle is configured to collect liquid in said respiratory circuit resulting from trapped moisture.

14. A method for collecting moisture in a respiratory airline system comprising:
configuring a moisture trap system inline in a patient circuit of said respiratory airline system;
connecting a receptacle to said moisture trap system; and
collecting liquid in said respiratory circuit resulting from trapped moisture.

15. The method of claim 14 wherein said moisture trap system comprises:
an inlet tube in fluid connection with an outlet tube;
an annular lid comprising an airtight seal; and
a receptacle removably attached said annular lid.

16. The method of claim 15 wherein connecting a receptacle to said moisture trap system further comprises:
opening said airtight seal of said annular lid with an actuating member.

17. The method of claim 16 further comprising at least one of:
removing said receptacle;
emptying said receptacle;
cleaning said receptacle; and
replacing said receptacle with a new receptacle.

18. The method of claim 17 wherein removing said receptacle further comprises:
automatically closing said airtight seal of said annular lid.

19. The method of claim 14 further comprising:
adapting said inlet tube to fit said patient circuit; and
adapting said outlet tube to fit said patient circuit.

20. The method of claim 19 further comprising:
securing said inlet tube and said outlet tube to said patient circuit with a banding assembly.
